(19) United States
(12) Patent Application Publication
(10) Pub. No.: US 2002/0192239 A1
(43) Pub. Date: Dec. 19, 2002

(54) Use of botulinum toxin for the treatment of chronic facial pain

(76) Inventors: Gary E. Borodic, Canton, MA (US); Martin A. Acquadro, Wellesly, MA (US)

Correspondence Address: Milbank, Tweed, Hadley & McCloy LLP, 1 Chase Manhattan Plaza, New York, NY, 10005-1413, US

(21) Appl. No.: 10/040,830

(22) Filed: Jan. 8, 2002

Related U.S. Application Data
(60) Provisional application No. 60/260,515, filed on Jan. 9, 2001.

(51) Int. Cl. A61K 39/08

(52) U.S. Cl. 424/247.1

(57) ABSTRACT

The present invention includes a method of treating pain caused by neuralgia comprising administering botulinum toxin to an afflicted area of a patient. The pain may be caused by trigeminal neuralgia or be associated with dental extraction or reconstruction, and may be facial pain. The neuralgia may be associated with compressive forces on a sensory nerve, intrinsic nerve damage, demyelinating disease, a genetic disorder, a metabolic disorder, central neurologic vascular disease, or trauma. The present invention also includes a method of treating post-operative incisional wound pain comprising administering botulinum toxin to an afflicted area of a patient. The post-operative incisional wound pain may be associated with medical treatments selected from the group consisting of sinus surgery, removal of an eye, temporal mandibular joint surgery, parotid gland extraction and resection, craniotomy for removal of an intracranial tumor, intra-ocular surgery, acoustic neuroma surgery, reconstructive procedures after tumor resection, radiation therapy for the treatment of cancer, skull base surgery, orbitectomy, facial bone removal, muscle removal, skin removal, and construction of myocutaneous flaps.

This application claims the benefit of U.S. Provisional Application Ser. No. 60/260,515, filed Jan. 9, 2001.

### BACKGROUND OF THE INVENTION

Chronic facial pain caused by neuralgia often presents difficult management problems requiring interdisciplinary consultations and multiple attempts at different therapy modalities. In a recent study, a series of chronic facial pain patients deemed to be ineffectively controlled with conventional medical and surgical procedures were treated with transcutaneous injection of botulinum type A toxin. The cases were classified into the following categories: 1. idiopathic trigeminal neuralgia; 2. post surgical chronic pain syndromes. The trigeminal neuralgia patients included who failed medical therapy and did not go on to have a surgical procedure, and those who failed medical therapy but later had temporary relief for a variable duration following a surgical procedure, only to have their pain return.

Botulinum toxin has been used extensively to treat regional dystonias which are often associated with pain or some form of sensory disturbance. Botulinum toxin injections for the treatment of spasmodic torticollis repeatedly demonstrated efficacy mitigating pain at rates substantially greater than other components of this syndrome (see references 1-3). Such observations led to the study of non-dystonic pain syndromes, such as myofascial pain and tension headache, which initially produced beneficial results (reference 4) and is currently being studied in larger controlled trials. Furthermore, trial injections after skull base surgery further indicated potential efficacy in a small series of patients. Recently, botulinum toxin is being investigated for the treatment of migraine headache (reference 5) and initial blinded controlled studies have produced some evidence of efficacy. The mechanisms involved in migraine headache have been reviewed (see references 20, 21), and have been noted to be vascular in origin.

Because of the long duration of action and the general lack of serious systemic effects associated with the use of type A botulinum toxin, a pilot trial described herein targeted the treatment of chronic facial pain patients who failed conventional therapeutic modalities and were seeking care from a tertiary pain clinic. This open label single site trial involved dosing units conventionally applied for other facial applications, such as essential blepharospasm, hemifacial spasm, and the treatment of myofascial pain (see references 6-9, 11).

### SUMMARY OF THE INVENTION

The present invention includes a method of treating pain caused by neuralgia comprising administering botulinum toxin to an afflicted area of a patient. The pain may be caused by trigeminal neuralgia or be associated with dental extraction or reconstruction, and may be facial pain. The neuralgia may be associated with compressive forces on a sensory nerve, intrinsic nerve damage, demyelinating disease, a genetic disorder, a metabolic disorder, central neurologic vascular disease, or trauma.

The present invention also includes a method of treating post-operative incisional wound pain comprising administering botulinum toxin to an afflicted area of a patient. The post-operative incisional wound pain may be associated with medical treatments selected from the group consisting of sinus surgery, removal of an eye, temporal mandibular joint surgery, parotid gland extraction and resection, craniotomy for removal of an intracranial tumor, intra-ocular surgery, acoustic neuroma surgery, reconstructive procedures after tumor resection, radiation therapy for the treatment of cancer, skull base surgery, orbitectomy, facial bone removal, muscle removal, skin removal, and construction of myocutaneous flaps.

### DETAILED DESCRIPTION OF VARIOUS EMBODIMENTS OF THE INVENTION

Patients treated at the Massachusetts General Hospital and Massachusetts Eye and Ear Infirmary for chronic facial pain were evaluated for possible botulinum toxin injections. Each patient was questioned for any past history of neuromuscular disease, which would potentially contraindicate the use of botulinum toxin, such as myasthenia gravis (see reference 10). Informed consent was obtained with emphasis on transient attendant risk of weakness and disfigurement produced by the localized injections of botulinum toxin.

Criteria for entry into the study were: failure of at least three medicinal therapies; description of high severity and intensity of pain; ability of the patient to describe a distinct anatomic area over which the pain was experienced; presence of the problem for at least two years; and understanding of the informed consent with emphasis on transient facial weakness.

Botulinum type A toxin was obtained in a lyophilized form and reconstituted with preservative free normal saline for injection at a concentration of 5 LD 50 units per 0.1 ml. One LD 50 unit is the dose necessary to kill 50% of population of 20-30 gm Swiss-Webster mice. Effort was made to limit initial exposure to a total dose of less than 50 units so as to avoid inducing muscular weakness, however, in severe situations, as much as 200 units can be used.

Injection sites were chosen according to the patient's descriptive and demonstrative anatomic localization of the pain. Brow and upper or lower eyelid injections were avoided as injection sites to avoid diffusion of toxin into the orbit, as ptosis and diplopia may result.

Injections were tailored to the location of the pain and can be generally summarized as follows:

1. Post-surgical Incisional Wound Pain: Botulinum is injected to the region where pain is localized, usually close to the surgical incision sites. If failing to achieve effect, a second injection would be given more diffusely throughout the dermatome in which the incision was made or to contiguous dermatomes.

2. Trigeminal neuralgia: Multifocal injections are given over the dermatome where pain was experienced.

Multiple surface injections may be used to disperse and affect as many sensory nerve endings as possible within the receptive field of a sensory neuron. A standard dermatome chart may be used to assess the level of spinal sensory nerves targeted for treatment.

Injections were given generally 10 mm apart so as to cover the anatomic region in which the facial pain was experienced. The injection depth varied between one and three millimeters. Each patient was assessed with CT or MRI to rule out the presence of structural pathology.

Each patient's diagnosis was confirmed by at least two physicians. An effort was made to characterize each patient's pain as neuropathic, myopathic, or combined neuropathic-myopathic in quality. For the purposes of this study, neuropathic pain was defined as localized over a division of the trigeminal nerve, having a sharp stabbing quality, and an ‘on-off’ temporal quality precipitated or made worse by touch. Such a description would be characteristic of typical trigeminal neuralgia. Myopathic pain is defined as a dull aching type of pain with palpable trigger points, often improved by tactile stimulation, and associated with soreness and pain radiation to the posterior cervical area upon mouth opening. This distinction was made to insure characterization of trigeminal neuralgia as a neuropathic process and not myopathic. Patients having an evolution of a syndrome type over a period of years received classification and diagnosis at the time of treatment rather than based upon symptomatology at the onset of pain.

Patients were followed at 2 and 6 weeks after treatment to assess the outcome. Improvement was categorically defined as a either a perception by the patient of at least 50% reduction in frequency and/or intensity of pain, or reduction in use of any analgesic medication, or expressed desire for further injections, with the patient acknowledging definite benefit following the first injection.

Data were treated categorically with respect to outcome.

The nosological diagnosis for the patients studied are given in Table 1.

Total dose received per injection cycle ranged between approximately 10 and 200 LD 50 Units (average 48.3 units), with a maximum of 7.5 units per percutaneous puncture. In each diagnostic category there was a substantial number of responders, including patients with trigeminal neuralgia. The responses varied from partial relief of pain (>50% improvement of pain as stated by the patient) to complete relief of pain. The duration of effect varied from 5-12 weeks, which is consistent with the known duration of action of botulinum toxin for other indications. No diagnostic category demonstrated any significant difference in response rates, although many of the patients with trigeminal neuralgia did experience partial responses.

In patients diagnosed with trigeminal neuralgia, the average duration of disease was 10 years (range 3-37 years) and all patients failed conventional medical therapy with carbamazepine and phenytoin. Other medical or surgical interventions also failed: three patients underwent radiofrequency ablation of the trigeminal nerve, four patients underwent chemical ablation (phenol, glycerol, or alcohol), and one patient underwent craniotomy with microvascular decompression (see references 12, 15-19).

Patients in the study experienced post-operative pain caused by the following surgical procedures: enucleation, orbitectomy, endoscopic and traditional sinus procedures, temporal mandibular joint reconstruction, large facial plastic reconstruction following cancerous tongue resections, trans-occipital craniotomy for the resection of acoustic neuroma or other skull base tumors, and dental extraction. No patient was treated within 3 months of the surgical procedure, and all patients had experienced sustained pain for at least 3 months.

Complications experienced by the patients included transient facial asymmetry during dynamic movements. No patient experienced systemic side effects.

While chronic facial pain can be a difficult management problem, in this study various types of chronic facial pain were identified and effectively treated with injection of botulinum toxin into involved areas achieving total or partial relief of symptoms, without the use of further systemic medications carrying notable side effects. The long duration of action of botulinum toxin and highly limited systemic complications associated with its use are attractive pharmacological attributes of this therapy for the management of these chronic pain syndromes. Complications are limited to possible temporary regional weakness over the injection sites and asymmetry of facial expression during dynamic facial movements. Although occasionally annoying, patients generally found these complications tolerable, considering the gravity of the affliction for which treatment was being sought, particularly for trigeminal neuralgia.

Neuralgia-related facial pains result from a variety of common etiologies, or are associated with a diversity of disease states; however, all the precise mechanisms responsible for the pains are more often not completely understood or even known.

The sensory innervation of the face is predominantly supplied by the trigeminal nerve. The trigeminal sensory complex describes the multiple connections and communications between the trigeminal nucleus and other nervous system regions: other cranial nerve nuclei; the reticular activating system; the autonomic nervous system; the thalamus; and multiple other ascending and descending nervous system atracts which facilitate or suppress excitatory and inhibitory pathways from above and below the brainstem area (see reference 12). Furthermore, the trigeminal sensory complex dips down as a continuum into the dorsal horn of the spinal cord to a level of approximately C4. This helps explain the convergence and referral patterns so often seen in neuralgia-related facial pain.

The basic science and clinical pain literature has established the great importance of discovery and recognition of the mechanisms causing pain. Indeed, a number of disease etiologies can share common mechanisms of pain. It is the fundamental importance of understanding the mechanisms of pain which dictates pain treatment. Knowing the etiology may often help prevent, alter, or cure a disease process, but the mechanisms of the pain may be independent of the etiology, known or unknown, or whether the etiology can be corrected (see reference 13, 14).

We have applied botulinum toxin to a number of patients with a variety of neuralgia-related facial pains involving a number of divergent etiologies. Systemic medications have historically been variable for these syndromes, and have not had any particular correlation or predictive value for outcome with other applications of botulinum toxin.

Botulinum toxin has been noted to effectively reduce or eliminate pain or aberrations in sensory experience associated with a number of diseases including adult onset spasmodic torticollis, bruxism, tension headache, myofascial pain, migraine, dystonic limb spasms, hemifacial contracture after seventh cranial nerve injury, and cervical spasms after skull base surgery. Furthermore, improvement in photophobia (reference 11) has been consistently noted in patients with essential blepharospasm and Meige syndrome.

A most remarkable clinical category in the presently-described study included patients with trigeminal neuralgia. This syndrome is difficult to treat and has an enormous negative impact on quality of life. The literature is lacking in double blind placebo control trials involving the use of many of the accepted first line systemic medications, such as carbamazepine (Tegretol), phenytoin (Dilantin), gabapentin (Neurontin), tricyclic antidepressants, and baclofen (see reference 16). The sustained efficacy of phenol, glycerol, or alcohol injections (reference 17) and thermocoagulation (reference 18) are not well documented. Dysesthesia and corneal numbness represent problematic eye complications associated with both thermocoagulation and phenol, glycerol, or alcohol injections. Microvascular decompression has been widely used for treatment of refractory cases with benefit reported at high percentages in a recently reported series (reference 19). However, attendant serious complications associated with intracranial surgery limits the application of this technique.

Aberrant blood vessels providing an irritating and scarring nidus for the intracranial portion of the trigeminal nerve has been a mechanism of pain, such that surgical lesion for therapeutic microvascular decompression has been tried. However, in general, it remains unknown how neuralgia is caused, and by what mechanism botulinum toxin exerts its therapeutic effect on neuralgia and post-operative incisional wound pain. The exact mechanisms by which botulinum toxin relieves such pain will be a subject of future study.

Botulinum toxin injections would offer some distinct advantages over existing therapies with respect to safety and efficacy. Weakness induced by botulinum toxin is transient, usually resolving within several weeks. Dosing levels per injection reported in this study are below levels used for conditions in which immunologic resistance has been commonly reported. The dose range varied from 10-200 units injected in 1-4 locations, but were most frequently effective at doses below 100 units, whereas doses ranging from 100-300 units have been used for spasmodic torticollis, the indication most commonly associated with antibody formation (see references 22) using Hall strain derived type A botulinum toxin preparations. The increase in specific activity in recent preparations has also apparently mitigated this complication.

In summary, patients with neuralgia-related chronic severe pain or post-operative incisional wound pain respond to botulinum toxin injections, thus providing a useful non-surgical tool for management of difficult cases. Facial weakness and impaired facial expression are the most common side effects. Facial weakness was most noticeable when the inner malar region of the face was targeted. Clearly further study of efficacy using double-blinded designs would be useful to advance these observations.

TABLE 1
Stratification of patient population by primary diagnosis and response

| | No of Patients | Responding |
|---|---|---|
| Total Treated | 61 | 46 |
| Neuralgia- | 23 | 18 |
| Trigeminal | | |
| Post Operative | 32 | 25 |
| Facial Pain Syndrome | | |
| Post Dental Surgery | 6 | 3 |

TABLE 3
Migraine history in remote past:

| | No of Patients | Responding |
|---|---|---|
| Migraine | 11 | 8 |
| History | | |
| No migraine | 34 | 25 |
| History | | |

Thus, presence or absence of history of migraine is not linked to patient responsiveness to botulinum toxin treatment for neuralgia or post-operative incisional wound pain.

Data derive from a cohort of patients presenting with essential headache, temporal mandibular joint syndrome, trigeminal neuralgia, and post incision (post operative wound pain).

Migraine is defined as severe throbbing headaches with nausea or vomiting associated with either photophobia, photophobia, or worsening with physical activity.

References

1. Borodic GE, Mills L, Joseph M. Botulinum A toxin for adult onset spasmodic torticollis. Plastic and Reconstructive Surgery 87:2, 285-289, 1991.

2. Poewe W, Wissel J. Use of botulinum toxin in the treatment of cervical dystonia. Bailleres Clin Neurol 1993 April 2(1):179-85.

3. Boghen D, Flanders M. Effectiveness of botulinum toxin in the treatment of spasmodic torticollis. Eur. Neurol. 1993 33(3): 199-203.

4. Acquardro M, Borodic GE. Treatment of myofascial pain with botulinum toxin. Anesthesiology 80(3): 705-706, 1994.

5. Silberstein S, Mathew N, Saper J Jenkins S. Botulinum toxin type A as a migraine preventive treatment. Headache 2000 June 40(6) :445-50.

6. Borodic, G. E., Cozzolino, D. Blepharospasm and its treatment with emphasis on the use of botulinum A toxin. Plastic & Reconstructive Surgery. vol. 83, no. 3:546-553, 1989.

7. Johnson EA. Clostridial toxins as therapeutic agents : benefits of natures most toxic proteins. Ann. Rev. Microbiol. 1999 53:551-75.

8. Mauriello JA Keswani R Franklin M. Long term enhancement of botulinum toxin injections by upper eyelid surgery in 14 patients with facial dyskinesia. Arch. Otolaryngol. Head Neck Surg. 1999 125 (6) 627-31.

9. Anderson RL, Patel BC Holds JB Jordan DR. Blepharospasm past, present and future. Ophthal. Plast. Reconstr. Surgery 1998 14(5): 305-17.

10. Borodic GE. Myasthenia gravis after botulinum toxin. 1998 Lancet 5: 352 (9143) 1832.

11. Borodic GE. Photophobia, an unrecognized component of the essential blepharospasm syndrome. Assoc. of Ophthalmic Plastic and Reconstructive Surgery, 1997 Annual Meeting Presentation.

12. Sessle BJ. Neural mechanisms of oral and facial pain. Otolaryngologic Clinics of North America 1989; 22(6): 1059-72.

13. Woolf CJ, Mannion RJ. Neuropathic pain: aetiology, symptoms, mechanisms, and management. Lancet 1999; 353: 1959-64.

14. Woolf CJ, Costigan M. Transcriptional and posttranslational plasticity and the generation of inflammatory pain. Proc. Natl. Acad. Sci. 1999; 96: 7723-30.

15. Costigan M, Woolf CJ. Pain: molecular mechanisms. The Journal of Pain; Supplement 1, 2000; 1: 35-44.

16. Fields HL. Treatment of trigeminal neuralgia. N. Engl. J. Med. 1996 April 25:334(17), 1125-6.

17. Wilkinson HA. Trigeminal nerve peripheral branch phenol/glycerol injections for tic douloureux. J. Neurosurg. 1999 90(5):828-32.

18. Yoon KB Wiles JR Miles JB Nurmikko TJ. Long term outcome of percutaneous thermocoagulation for trigeminal neuralgia. Anesthesia 1999 54(8)803-808.

19. Barker F Jennetta P Bissonette P et al. The long term outcome of microvascular decompression for trigeminal neuralgia. N. Engl. J. Med. 1996 334:1077-83.

20. Buzzi MG Dimitriadou V Theoharides TC Moskowitz MA Moscowitz. 5-hydroxytryptamine receptor agonists for the abortive treatment of vascular headaches block mast cell, endothelial and platelet activiation within the rat dura mater after trigeminal stimulation. Brain Res. 1992 June 26:528 (1-2); 127.

21. Buzzi MG Bonamini M Moskowitz MA. Neurogenic model for migraine. Cephalgia 1995 15 (4): 277-289.

22. Borodic GE, Johnson, Goodnough M, Schantz E. Botulinum toxin, Immunologic resistance, and problems with available materials.(by invitation), 46:26-29 Neurology, 1996.

What is claimed is:

1. A method of treating pain caused by neuralgia comprising administering botulinum toxin to an afflicted area of a patient.

2. The method of claim 1, wherein the pain is caused by trigeminal neuralgia.

3. The method of claim 1, wherein the pain is facial pain.

4. The method of claim 1, wherein the neuralgia is associated with compressive forces on a sensory nerve.

5. The method of claim 1, wherein the neuralgia is associated with intrinsic nerve damage, demyelinating disease, or a genetic disorder.

6. The method of claim 1, wherein the neuralgia is associated with a metabolic disorder.

7. The method of claim 1, wherein the neuralgia is associated with central neurologic vascular disease.

8. The method of claim 1, wherein the neuralgia is associated with trauma.

9. The method of claim 1, wherein the botulinum toxin is administered in a dose of from 10 to 200 LD 50 units.

10. The method of claim 1, wherein the pain is associated with dental extraction or reconstruction.

11. The method of claim 1, wherein the botulinum toxin is selected from the group consisting of immunotypes A-G.

12. A method of treating post-operative incisional wound pain comprising administering botulinum toxin to an afflicted area of a patient.

13. The method of claim 12, wherein the pain is associated with medical treatments selected from the group consisting of sinus surgery, removal of an eye, temporal mandibular joint surgery, parotid gland extraction and resection, craniotomy for removal of an intracranial tumor, intra-ocular surgery, acoustic neuroma surgery, reconstructive procedures after tumor resection, radiation therapy for the treatment of cancer, skull base surgery, orbitectomy, facial bone removal, muscle removal, skin removal, and construction of myocutaneous flaps.

13. The method of claim 12, wherein the botulinum toxin is selected from the group consisting of immunotypes A-G.

14. The method of claim 6, wherein the metabolic disorder is selected from the group consisting of diabetes and amyloidosis.
